(19) United States
(12) Patent Application Publication
(10) Pub. No.: US 2011/0137258 A1
(43) Pub. Date: Jun. 9, 2011

(54) NASAL RINSE APPARATUS

(76) Inventor: Kambiz YADIDI, Los Angeles, CA (US)

(21) Appl. No.: 12/634,681

(22) Filed: Dec. 9, 2009

(51) Int. Cl. A61M 3/02

(52) U.S. Cl. 604/181

(57) ABSTRACT

A nasal rinse apparatus is described, including a container having an upper surface, at least a first portion of the container tapers from proximally to distally to a maximum height at the upper surface, at least a second portion of the container tapers from proximally to distally to a minimum height at the upper surface and portions in between tapers from to a height at the upper surface that is between the maximum and minimum height. In some embodiments, a dispenser having an aperture is configured to deliver a fluid from the container into a nasal cavity of a user at a jet trajectory axis between about 80 degrees and about 100 degrees with respect to a principle axis of a container connection portion.

### FIELD

The subject technology generally relates to nasal and sinus rinse procedures and apparatus and, in particular, to a nasal rinse system.

### BACKGROUND

Existing sinus rinse devices usually include a tube in an upright dispenser, or irrigator, to siphon solution from the bottom of the upright dispenser, through an opening in the top of the upright dispenser as a user squeezes the sides of the upright dispenser during nasal irrigation.

### SUMMARY

Nasal and nasopharyngeal cavities are common places for holding environmental allergens and contaminants, such as pollen, fungal spores, animal body-originating dustings, dust, soot, ash, bacteria, viruses and the like. A nasopharyngeal system protects the nasal system by trapping such contaminants and allergens. These contaminants and allergens may be trapped by a system of mucous lined passageways. These contaminants and allergens may cause allergic reactions and other health consequences. The nasopharyngeal system is not self-cleaning and as a result, many disease-causing agents such as contaminants or allergens exist in the nasal and nasopharyngeal cavities. Hence, a need efficient means for cleaning the nasopharyngeal system.

As used herein, the term “sinus rinse device” is defined broadly and can include devices that may rinse or supply fluid to a nasal cavity, nasopharynx, oropharynx, oral cavity, or one or more paranasal sinuses. The technique of using a tube to siphon a solution from a solution is undesirable because it does not allow the user to completely empty the dispenser during normal use. Further, the tube is an additional component that can reduce the overall reliability and functional life of the dispenser. For example, the dispenser cannot be operated by the user as intended if the tube becomes defective. An additional process is required to detach, clean, and re-attach the tube to the sinus rinse product. The tube is susceptible to damage during the detaching, cleaning, and/or re-attaching process thereby rendering the sinus rinse product non-functional.

Further, the tube is a natural trap in which bacteria and/or mold can colonize and potentially infect the user. It is not uncommon for black mold to accumulate in the tube of the sinus rinse product and the possibility of pseudomonas bacteria developing in the tight-fitting areas where the tube attaches to a nozzle of the sinus rinse product is of concern to some physicians. Furthering the risk is the chance of some users not cleaning the tube with any frequency thereby increasing the chances for infection with each subsequent use. Furthermore, due to the Venturi Effect created by the tube on such sinus rinse products, the reflux action caused by the re-expansion of the dispenser, causes upper respiratory secretions including infected materials to be siphoned from the patient's nasal passages, back into the patient as they continue to irrigate their nasal cavity.

In one aspect of the disclosure, a nasal rinse apparatus may comprise a container and a dispenser. The container may comprise flexible walls or sidewalls having a proximal section and a distal section. The distal section configured to terminate at an upper surface. At least a first portion of the container tapers from proximally to distally to a maximum height at the upper surface and at least a second portion of the container tapers from proximally to distally to a minimum height at the upper surface. Other portions of the container, between the first and second portions, taper from proximally to distally to a height at the upper surface that is between the maximum height and the minimum height. A container connection portion may be disposed at the upper surface of the container. The container connection portion comprises a container connection cavity defining a principal connection axis. The container connection portion also defines an inclination angle between the principal connection axis and a primary long axis of the container. The primary long axis extends through the proximal and distal sections of the container. The dispenser comprises an interior cavity in fluid communication with the container, a proximal opening and a distal opening. The distal opening forms an aperture configured to deliver a fluid from the container into a nasal cavity of a user. A dispenser connection portion is at a proximal region of the dispenser where the dispenser connection portion is configured to couple to the container connection portion. When fluid in the container is ejected from the dispenser through the aperture, the fluid forms a jet trajectory primarily along a jet axis. The jet axis forms an angle of between about 80 and about 100 degrees with respect to the principle connection axis of the container connection portion.

Some embodiments include a nasal rinse apparatus comprising container, having flexible sidewalls and comprising a proximal section and a distal section; wherein the distal section terminates at an upper surface, at least a first portion of the container tapers from proximally to distally to a maximum height at the upper surface, at least a second portion of the container tapers from proximally to distally to a minimum height at the upper surface; wherein other portions of the container, between the first and second portions, taper from proximally to distally to a height at the upper surface that is between the maximum height and the minimum height; a dispenser disposed at the distal end, comprising an interior cavity in fluid communication with the container, a proximal opening and a distal opening, the distal opening forming an aperture configured to deliver a fluid from the container into a nasal cavity of a user; and wherein, when fluid in the container is ejected from the dispenser through the aperture, the fluid forms a jet trajectory primarily along a jet axis, the jet axis forming an angle of between about 70 degrees and about 110 degrees with respect to a principle axis orthogonal to the upper surface.

In some embodiments, the angle is between about 80 degrees and about 100 degrees. In some embodiments, the angle is about 90 degrees.

In some embodiments, the container further comprises a container connection portion disposed at the upper surface, the dispenser further comprises a dispenser connection portion, wherein the container connection portion is in fluid communication with, and couples with, the dispenser connection portion.

In some embodiments, the dispenser connection portion has a substantially circular cross section having an outermost circumference, and wherein at least part of the dispenser extends laterally beyond the circumference. In some embodiments, a latitudinal cross section of the aperture is eccentrically positioned with respect to the circumference.

In some embodiments, the nasal rinse apparatus is configured to have the dispenser positioned lower than the container during delivery of the fluid from the container into the nasal cavity of the user.

In some embodiments, when the nasal rinse apparatus is configured such that when the dispenser is positioned lower than the container, fluid flows through the aperture due to gravity.

In some embodiments, when fluid is in the container and when the flexible sidewalls are squeezed such that fluid pressure rises in the container, the fluid is ejected through the aperture.

In some embodiments, an inclination angle between the principal axis and a primary long axis of the container is between about 5 and about 70 degrees.

In some embodiments, an inclination angle between the principal axis and a primary long axis of the container is between about 20 and about 30 degrees.

In some embodiments, an inclination angle between the principal axis and a primary long axis of the container is about 25 degrees.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology.

Accordingly, the drawings and detailed description are to be regarded as illustrative in nature and not as restrictive.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 is a diagram illustrating an example of a perspective view of s nasal rinse apparatus according to one aspect of the subject technology.

FIG. 2 is an example of a sectional view of a container of the nasal rinse apparatus.

FIG. 3 is an example of a plan view from the top of the container.

FIG. 4 is an example of a sectional view of a dispenser of the nasal rinse apparatus.

FIG. 5 illustrates an example side view of the ornamental features of the dispenser.

FIG. 6 is an example of a plan view from the top of the dispenser.

FIG. 7 illustrates an example of an application of the nasal rinse device to the nasal cavity of a user.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like or similar components may be labeled with identical element numbers for ease of understanding or it may indicated in the disclosure that one component may be an example of a different component.

By way of illustration and not limitation, aspects of the subject technology describe a nasal rinse apparatus. One aspect of the subject technology comprises a nasal rinse device comprising a container and a dispenser coupled to the container, where the dispenser comprises an opening for channeling a fluid to a nasal cavity of a user. The container may be filled with the fluid such as water and a saline mixture while the container is in a traditional upright position. In some configurations, the dispenser is an integral part of the container. In other configurations, the dispenser is independent of the container. During nasal irrigation, for example, the nasal rinse apparatus is inverted with the opening facing in an angled, downward position.

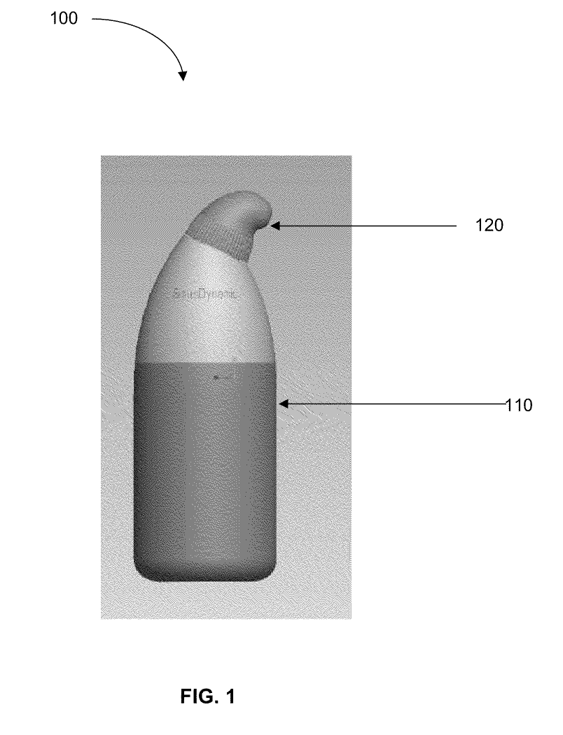

Referring now to the illustrations, and more particularly to FIG. 1, an example of a nasal rinse apparatus 100 according to one aspect of the subject technology is shown. In general, the nasal rinse apparatus 100 may comprise a container 110 and a dispenser 120.

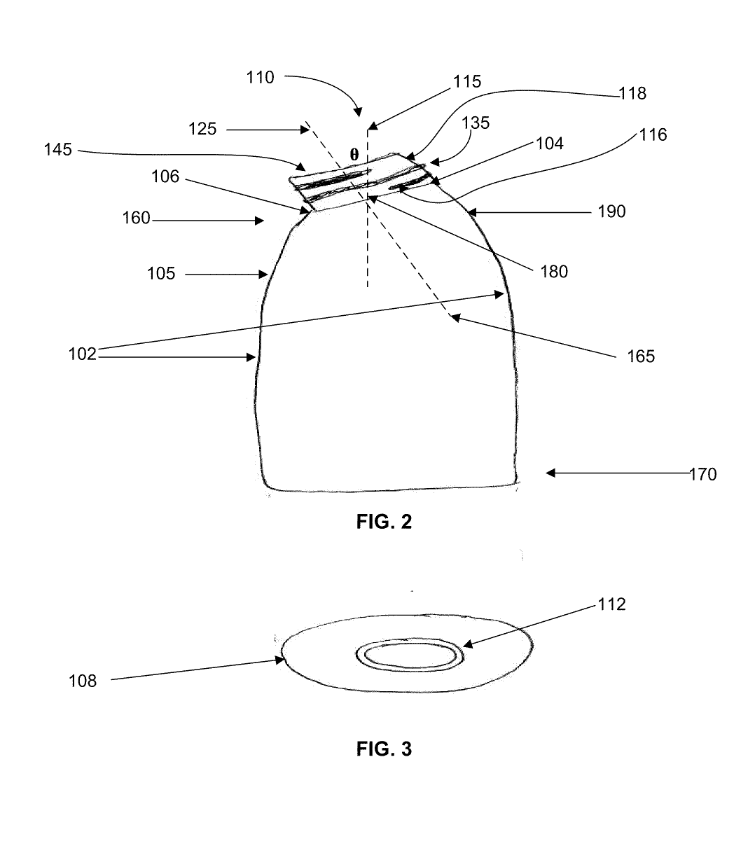

Referring to FIG. 2, the container 110 may comprise flexible sidewalls 102 and a proximal section 170 and a distal section 160. The distal section 160 may be configured to terminate at an upper surface 180. In some aspects of the subject technology, at least a first portion 190 of the container 110 tapers from proximally to distally to a maximum height at position 104 at the upper surface 180. At least a second portion 105 of the container 110 tapers from proximally to distally to a minimum height at position 106 at the upper surface 180 of the container 110. Other portions of the container 110 between the first portion 190 and the second portion 105 taper from proximally to distally to a height at the upper surface 180 that is between the maximum height at position 104 and the minimum height at position 106. The upper surface 180 defines a central axis 125, where the central axis 125 forms a termination or inclination angle θ between the central axis 125 and a primary long axis 115 of the container 110. The top view of a cross section 112 of the upper surface 180 indicates that the upper surface is centrally located or concentrically positioned with respect to a non-tapered cross section 108 of the container 110 (see FIG. 3). In some configurations, the cross section 112 may be eccentrically positioned. The primary long axis 115 extends through the proximal and distal sections 170 and 160 of the container 110. A container connection portion 135 comprising a container connection cavity 145 in fluid communication with the container 110 may be disposed at the upper surface 180. The container connection portion 145 defines a principal connection axis 115 through its longitudinal cross section. The container connection portion 135 defines a principal connection axis 165 that is coaxial with the central axis 125. Therefore, the container connection portion 135 also defines the inclination angle θ between the principal connection axis 165 and the primary long axis 115 of the container 110. In some configurations, the inclination angle between the principal connection axis 165 and a primary long axis 115 of the container 110 is between 5 and 70 degrees. In some configurations, the inclination angle θ between the principal connection axis 165 and a primary long axis 115 of the container 110 is between 20 and 30 degrees. In yet another configuration, the inclination angle θ between the principal connection axis 165 and a primary long axis 115 of the container 110 is 25 degrees. The container 110 functions as a reservoir for fluid to be dispensed into the dispenser 120 and subsequently to the nasal cavity of a user. In some configurations the dispenser is an integral part of the container. The fluid may comprise at least one of water, a saline mixture and any nasal cleansing fluid. The container connection portion 135 may comprise threads 116 or may comprise other connection or attachment means for connecting or attaching the container 110 to the dispenser 120. In some configurations, the threads 116 may be disposed on an outer surface 118 of the container connection portion 135. In some configurations, the threads may be disposed on an inner surface of the container connection portion 135. The container may comprise a high-density material such as natural or colored polyethylene or high-density polyethylene. In some configurations, the container may be manufactured or processed by blow molding. In some configurations, the weight of the container may be about 25 grams and the container can accommodate a capacity volume of between 236 milliliters and 244 milliliters.

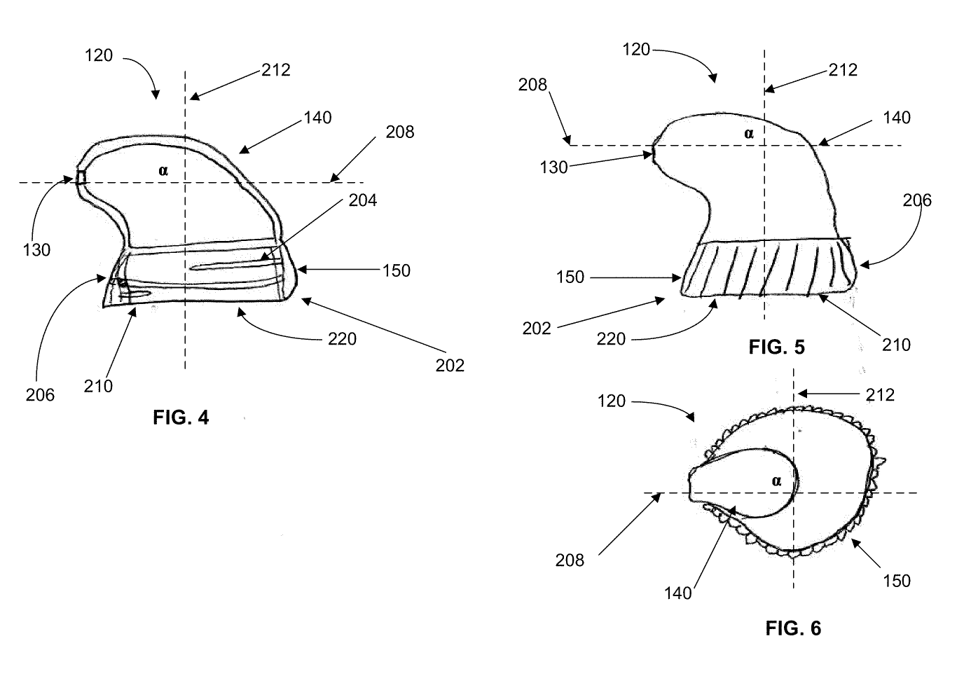

Referring to FIGS. 4, 5 and 6, the dispenser 120 comprises an interior cavity 220 in fluid communication with the container 110, a proximal opening 210 and a distal opening 130. The distal opening 130 forms an aperture (e.g. nasal aperture) configured to deliver the fluid from the container 110 into a nasal cavity of a user. The aperture 130 can be maintained in a non-coaxial alignment with the proximal opening 210. The flexible sidewalls 102 of the container 110 are configured to be squeezed to facilitate ejection of the fluid through the aperture 130 during nasal irrigation. The dispenser 120 further comprises a dispenser connection portion 150 at a proximal region 202 of the dispenser 120 and a nozzle portion 140 above the dispenser connection portion 150. The nozzle portion 140 forms a dome shaped elbow between the dispenser connection portion 150 and the aperture 130. The nozzle portion 140 can also be optionally round, oval, square, rectangular, triangular, pentagonal, hexagonal, heptagonal, octagonal, or irregular in shape. The dispenser connection portion 150 can be configured to couple to the container connection portion 135. In some configurations, the dispenser connection portion 150 has a substantially circular cross section having an outermost circumference, and wherein at least part of the dispenser extends laterally beyond the circumference. In some configurations, the dispenser connection portion 150 has a substantially circular cross section having an outermost circumference, and wherein a latitudinal cross section of the aperture is eccentrically positioned with respect to the circumference. In some configurations, the nozzle portion 140 may be offset between 50 to 70 degrees from a latitudinal axis of the container 110. In some configurations, the offset between the nozzle portion 140 and the latitudinal axis of the container 110 is about 60 degrees. The dispenser connection portion 150 may comprise threads 204 or may comprise other connection or attachment means for connecting or attaching to the container 110. In some configurations, the threads 204 may be disposed on an outer surface 206 of the dispenser connection portion 150. In some configurations, the threads 204 may be disposed on an inner surface of the container connection portion 135. The dispenser connection portion 150 can be coaxially aligned with the container connection portion 135. The container 110 can be configured to receive the fluid and to dispense the fluid via the dispenser 120. Thus, the container connection portion 135 is in fluid communication with, and couples with, the dispenser connection portion 150. The fluid in the container 110 may be ejected from the dispenser 120 through the aperture 130. The fluid may form a jet trajectory primarily along a jet axis 208 with the jet axis 208 forming an angle α between about 70 and about 110 degrees with respect to the principle connection axis 212 of the dispenser connection portion 150. When the dispenser 120 is integrated into the container 110, there may not be a need for the dispenser connection portion 150 or the container connection portion 135. In some configurations, the jet axis forms an angle of between about 70 degrees and about 110 degrees with respect to a principal axis orthogonal to the upper surface. In some configurations, when the fluid in the container 110 is ejected from the dispenser 120 through the aperture 130, the fluid forms a jet trajectory primarily along a jet axis 208. The jet axis forms an angle of about 90 degrees with respect to the principle connection axis 212 of the dispenser connection portion 150. In some configurations, the dispenser 120 can be an integral part of the container 110. As illustrated in FIG. 4, a non-aligned section of the nozzle portion 140 may be eccentrically positioned with respect to a circular proximal region 202 of the dispenser 120. In some configurations, a latitudinal cross section of the nozzle portion 140 is eccentrically positioned with respect to the circular proximal region 202 of the dispenser 120. In some configurations, the latitudinal cross section may be horizontally aligned with a primary horizontal or latitudinal axis 208 of the nozzle portion 140. The dispenser may comprise a material such as natural or colored polypropylene. In some configurations, the dispenser may be manufactured or processed by injection molding. In some configurations, the weight of the container may be between 3.0 grams and 3.5 grams.

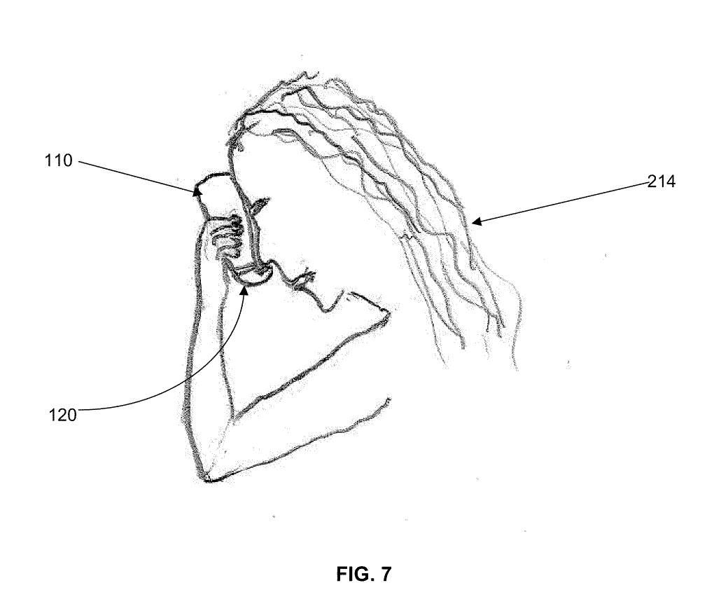

In preparation for use, the container 110 is filled with fluid by removing the dispenser 120 and pouring the fluid into the container 110 via the container connection cavity 145 while the container 110 is in the traditional upright position. When the appropriate amount of fluid has been poured, the dispenser 120 is replaced on the container 110. The nasal rinse apparatus 100 is inverted and positioned in an angled downward position as illustrated in FIG. 7 with the dispenser facing downward such that the aperture 130 is positioned to eject the fluid into the nasal cavity of the user 214. The angle of inversion may be between 35 and 55 degrees. In some configurations, the nasal rinse apparatus 100 is configured to have the dispenser 120 positioned lower than the container 110 during delivery of the fluid from the container into the nasal cavity of the user. In some configurations, the nasal rinse apparatus 100 is configured such that when the dispenser 120 is positioned lower than the container 110, fluid flows through the aperture 130 due to gravity. In some configurations, when fluid is in the container and when the flexible sidewalls are squeezed such that fluid pressure rises in the container, the fluid is ejected through the aperture.

The dispenser may be positioned to touch a portion of the nose and/or the forehead of the user 214 to facilitate nasal irrigation. In this position, the nasal rinse apparatus 100 ejects the fluid through the aperture 130 according to a force of gravity due to the inverted positioning of the nasal rinse apparatus 100. To facilitate dispensing of the fluid, the user 214 may squeeze the flexible walls 102 of the container 110 to dispense fluid from the container 110 through the dispenser 120 to the nasal cavity of the user 214. This implementation may allow for complete utilization of the fluid, easy control of volume administration through the squeezing of the flexible sidewalls 102 of the container 110 and better directional control during use by children and adults. After use the user 214 or patient may remove the dispenser 120, rinse both the dispenser 120 and container 110 in a cleaning fluid such as clean water, and let them air dry detached.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A nasal rinse apparatus comprising:
a container, having flexible sidewalls and comprising a proximal section and a distal section;
wherein the distal section terminates at an upper surface, at least a first portion of the container tapers from proximally to distally to a maximum height at the upper surface, at least a second portion of the container tapers from proximally to distally to a minimum height at the upper surface;
wherein other portions of the container, between the first and second portions, taper from proximally to distally to a height at the upper surface that is between the maximum height and the minimum height;
a dispenser disposed at the distal end, comprising:
an interior cavity in fluid communication with the container;
a proximal opening and a distal opening, the distal opening forming an aperture configured to deliver a fluid from the container into a nasal cavity of a user; and
wherein, when fluid in the container is ejected from the dispenser through the aperture, the fluid forms a jet trajectory primarily along a jet axis, the jet axis forming an angle of between about 70 degrees and about 110 degrees with respect to a principle axis orthogonal to the upper surface.

2. The apparatus of claim 1, wherein the angle is between about 80 degrees and about 100 degrees.

3. The apparatus of claim 1, wherein the angle is about 90 degrees.

4. The apparatus of claim 1, the container further comprising a container connection portion disposed at the upper surface, the dispenser further comprising a dispenser connection portion, wherein the container connection portion is in fluid communication with, and couples with, the dispenser connection portion.

5. The apparatus of claim 4, wherein the dispenser connection portion has a substantially circular cross section having an outermost circumference, and wherein at least part of the dispenser extends laterally beyond the circumference.

6. The apparatus of claim 4, wherein the dispenser connection portion has a substantially circular cross section having an outermost circumference, and wherein a latitudinal cross section of the aperture is eccentrically positioned with respect to the circumference.

7. The apparatus of claim 1, wherein the nasal rinse apparatus is configured to have the dispenser positioned lower than the container during delivery of the fluid from the container into the nasal cavity of the user.

8. The apparatus of claim 7, wherein, when the nasal rinse apparatus is configured such that when the dispenser is positioned lower than the container, fluid flows through the aperture due to gravity.

9. The apparatus of claim 1, wherein, when fluid is in the container and when the flexible sidewalls are squeezed such that fluid pressure rises in the container, the fluid is ejected through the aperture.

10. The apparatus of claim 1, wherein an inclination angle between the principal axis and a primary long axis of the container is between about 5 and about 70 degrees.

11. The apparatus of claim 1, wherein an inclination angle between the principal axis and a primary long axis of the container is between about 20 and about 30 degrees.

12. The apparatus of claim 1, wherein an inclination angle between the principal axis and a primary long axis of the container is about 25 degrees.
